(19) United States
(12) Patent Application Publication
(10) Pub. No.: US 2016/0279034 A1
(43) Pub. Date: Sep. 29, 2016

(54) CLEANSING BAR WITH POLYMER CORE

(71) Applicant: John Leo Thelen, SR., Eastover, SC (US)

(72) Inventor: John Leo Thelen, SR., Eastover, SC (US)

(21) Appl. No.: 14/996,395

(22) Filed: Jan. 15, 2016

Related U.S. Application Data
(60) Provisional application No. 62/137,986, filed on Mar. 25, 2015.

(51) Int. Cl. A61K 8/02; A61Q 19/10

(52) U.S. Cl. CPC A61K 8/0233; A61Q 19/10

(57) ABSTRACT

A cleansing bar is provided. The cleansing bar includes a solid soap with a hollow core. A solid object is disposed within the hollow core. The solid object is insoluble in water. The present invention may be used while taking a shower or bath to clean the body. Once the solid soap has substantially dissolved, the solid object is visible. The user may then redeem the solid object for a monetary value from the manufacturer.

### CROSS-REFERENCE TO RELATED APPLICATION

This application claims the benefit of priority of U.S. provisional application No. 62/137,986, filed Mar. 25, 2015, the contents of which are herein incorporated by reference.

### BACKGROUND OF THE INVENTION

The present invention relates to a cleansing bar and, more particularly, to a cleansing bar with a polymer core.

Soap is a salt of fatty acid. Consumers mainly use soaps as surfactants for washing, bathing, and cleaning. Soaps for cleansing are obtained by treating vegetable or animal oils and fats with a strongly alkaline solution. Fats and oils are composed of triglycerides; three molecules of fatty acids attach to a single molecule of glycerol. The alkaline solution, which is often called lye, brings about a chemical reaction known as saponification. In this reaction, the triglyceride fats first hydroluze into free fatty acids, and then these combine with the alkali to form crude soap: an amalgam of various soap salts, excess fat or alkali, water, and liberated glycerol (glycerin). The glycerin, a useful by-product, can remain in the soap product as a softening agent, or be isolated for other uses. Currently, unused portions of soap bars are thrown away and sent to landfills. Soap contaminates soils, and is therefore a pollutant.

As can be seen, there is a need for an improved soap bar that reduces waste.

### SUMMARY OF THE INVENTION

In one aspect of the present invention, a cleansing bar comprises: a solid soap comprising a hollow core; and a solid object disposed within the hollow core, wherein the solid object is insoluble in water.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown in use;

FIG. 2 is a cutaway view of an embodiment of the present invention shown in use;

FIG. 3 is a perspective view of an embodiment of the present invention;

FIG. 4 is a section view of the present invention along line 3-3 in FIG. 1;

FIG. 5 is a section view of an embodiment of the present invention shown in a partially worn condition;

FIG. 6 is section view of an embodiment of the present invention shown in an exposed condition; and

FIG. 7 is a perspective view of an embodiment of the present invention in an exposed condition.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a clear acrylic soap bar insert. The present invention may be used to save raw materials and can be redeemed for money or products. Currently, there are no other devices in use now for the prevention of waste in bar soap manufacturing. The insert of the present invention is recyclable and therefore prevents soil contamination in landfills.

The clear acrylic bar soap insert is placed in the bar soap. The soap bar is then used. After using the soap, the clear acrylic resin will show itself. Instead of discarding the insert it can be returned to the manufacturer for recycling. The acrylic bar can be returned to a grocery store or a non-profit business to redeem a set value as determined by the manufacturer.

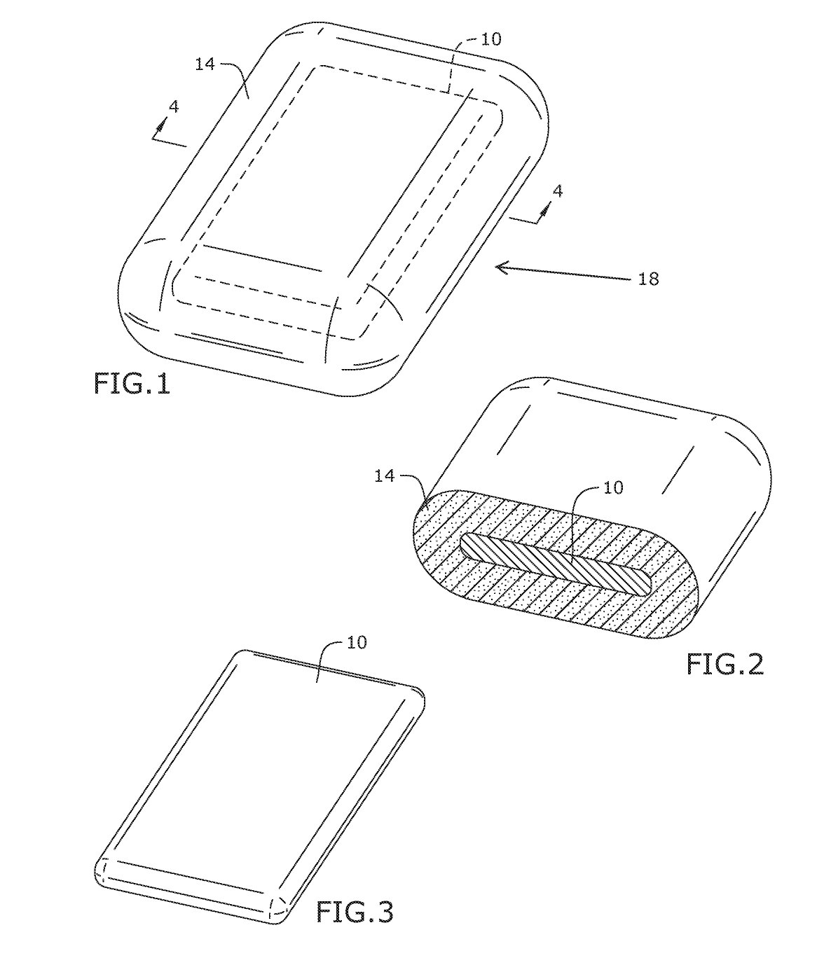

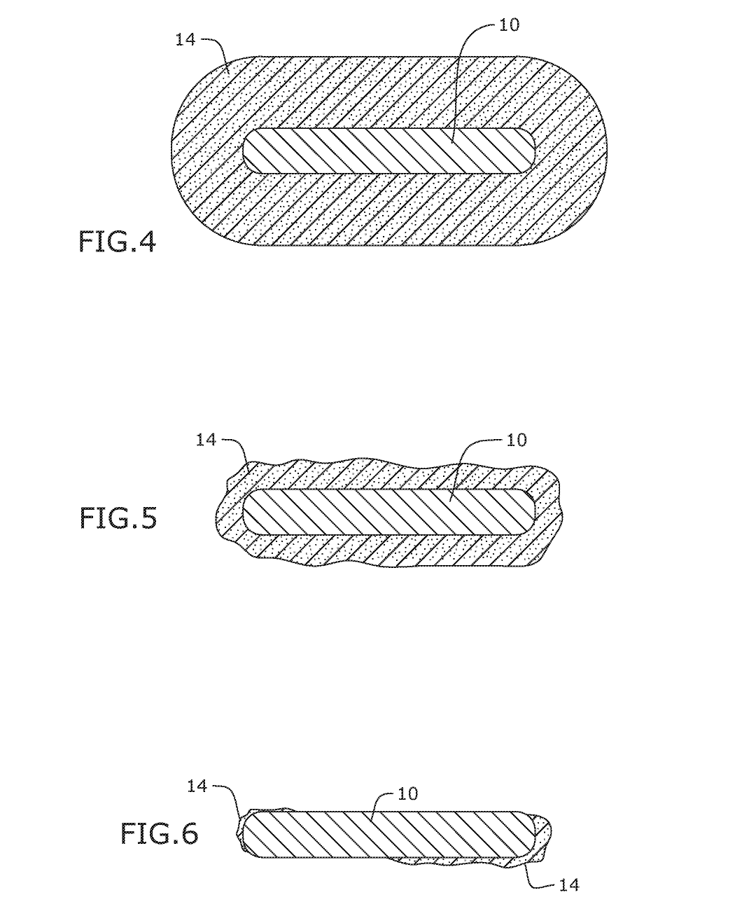

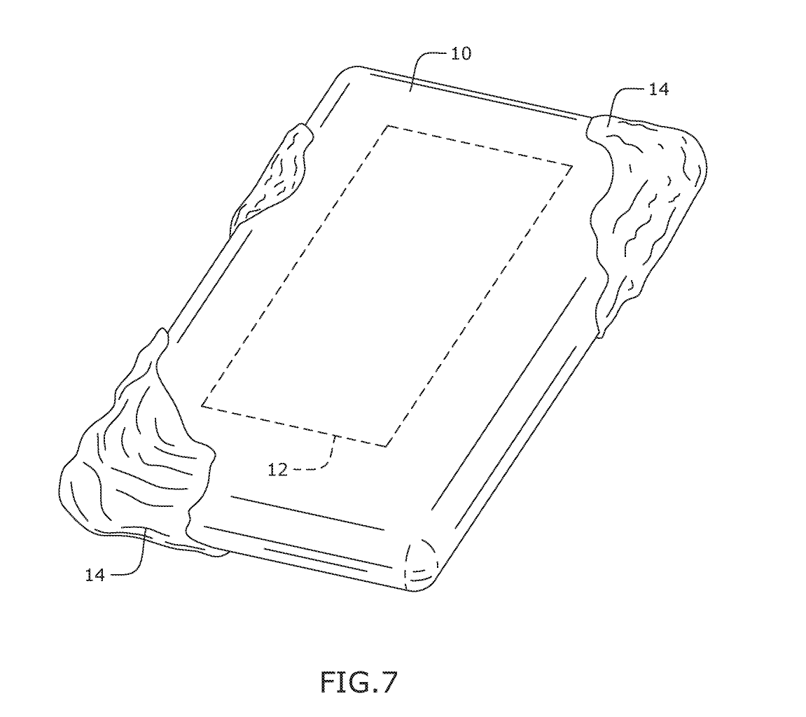

Referring to FIGS. 1 through 7, the present invention includes a cleansing bar 18. The cleansing bar 18 includes a solid soap 14 with a hollow core. A solid object 10 is disposed within the hollow core. The solid object 10 is not soap and is insoluble in water. The present invention may be used while taking a shower or bath to clean the body. Once the solid soap 14 has substantially dissolved, the solid object 10 is visible. The user may then redeem the solid object 10 for a monetary value from the manufacturer.

In certain embodiments, the solid object 10 is a rectangular shape, similar to the bar of soap. The solid object 10 may be a polymer, such as a plastic. The polymer may be an acrylic resin. In certain embodiments, the acrylic resin is translucent. In certain embodiments, an advertisement 12 may be stamped on an outer surface of the solid object 10. Therefore, the user may know which company to return the solid object 10 and redeem the monetary value.

To create the solid object, a clear acrylic resin and an appropriate curing solution are injected into a mold measuring approximately 64 mm long×32 mm wide and 5 mm thick. The solid object is cooled and solidifies. The solid object is then placed in a second mold comprising larger dimensions than the first mold. A hot liquid soap is then injected into the second mold and the hot liquid soap is allowed to cool, thereby turning into a solid bar of soap.

It should be understood, of course, that the foregoing relates to exemplary embodiments of the invention and that modifications may be made without departing from the spirit and scope of the invention as set forth in the following claims.

What is claimed is:

1. A cleansing bar comprising:
a solid soap comprising a hollow core; and
a solid object disposed within the hollow core, wherein the solid object is insoluble in water.

2. The cleansing bar of claim 1, wherein the solid object is a polymer.

3. The cleansing bar of claim 2, wherein the polymer is an acrylic resin.

4. The cleansing bar of claim 3, wherein the acrylic resin is translucent.

5. The cleansing bar of claim 1, further comprising an advertisement disposed on a surface of the solid object.

6. The cleansing bar of claim 1, wherein the solid object is rectangular shaped.
